(19) United States
(12) Patent Application Publication
(10) Pub. No.: US 2024/0090968 A1
(43) Pub. Date: Mar. 21, 2024

(54) SURGICAL ASSISTANCE SYSTEM HAVING SURGICAL MICROSCOPE AND CAMERA, AND REPRESENTATION METHOD

(71) Applicant: B. Braun New Ventures GmbH, Freiburg im Breisgau (DE)

(72) Inventor: Amir Sarvestani, Freiburg (DE)

(21) Appl. No.: 18/274,312

(22) PCT Filed: Jan. 31, 2022
(86) PCT No.: PCT/EP2022/052202

§ 371 Date: Jul. 26, 2023

(30) Foreign Application Priority Data
Feb. 1, 2021 (DE) 10 2021 102 274.6

(51) Int. Cl. A61B 90/20; A61B 34/20; A61B 90/00

(52) U.S. Cl. CPC A61B 90/20; A61B 34/20; A61B 90/361; A61B 2017/00123

(57) ABSTRACT

A surgical intervention system, image display method, storage medium, and sterile space. The system includes a microscope having a movable head with an optical system that magnifies a targeted area and produces a digital image with an image unit. A microscope arm connects to a base that adjusts a position and/or orientation of the microscope head. A control unit processes the image and controls a display device. The microscope further includes a camera system with a surroundings camera arranged at the microscope head. A field of view of the surroundings camera includes the field of view of the optical system to capture the targeted area and an environment around the targeted area and provide a surroundings image. The control device processes the surroundings image and generates a combined view with the microscope image and the surroundings image. The control device outputs the combined view on the display device.

### CROSS-REFERENCE TO RELATED APPLICATIONS

This application is the United States national stage entry of International Application No. PCT/EP2022/052202, filed on Jan. 31, 2022, and claims priority to German Application No. 10 2021 102 274.6, filed on Feb. 1, 2021. The contents of International Application No. PCT/EP2022/052202 and German Application No. 10 2021 102 274.6 are incorporated by reference herein in their entireties.

### FIELD

The present disclosure relates to a surgical assistance system comprising a surgical microscope/operation microscope for use in a surgical intervention/surgical procedure on a patient. The surgical microscope comprises a movable microscope head with a housing and an optical system, in particular provided in the housing, adapted to provide optical magnification of a targeted area (an intervention area) in the direction of an optical microscope axis, in particular at a distance between 5 cm to 80 cm, particularly preferably between 20 cm and 60 cm, and to create a (digital) microscope image via a microscope image unit, in particular comprising a sensor, and to provide it digitally/in a computer-readable manner. Furthermore, the surgical microscope comprises an articulatable/movable microscope arm provided at a base, on which the movable microscope head is arranged, in particular supported, and which is adapted to adjust a position and/or orientation of the microscope head in space. The surgical assistance system further comprises at least one display device for displaying a visual content, and a control unit adapted to process the microscope image and to control the display device for a view accordingly. In addition, the present disclosure relates to an image display method, a storage medium and a sterile space.

### BACKGROUND

Operation microscopes or surgical microscopes belong to the standard instruments used in surgical interventions, particularly in neurosurgery, spine surgery and microsurgery. These surgical microscopes are usually equipped with a binocular optical system, with which a surgeon can see the microscope picture/the microscopic picture/the microscope image directly optically magnified, or can be displayed in a magnified manner via a display device/an external notification device, such as an OR monitor, for the visual view of the (digitally) recorded microscope picture. In both cases, however, the surgeon sees only the view of the microscope picture of a targeted intervention area with very high magnification through the binoculars or the OR monitor. As a consequence, the surgeon is not or at least only with difficulty able to see an operation area/operation field/intervention area surrounding (the microscope picture) in order to locate his hands or medical instruments used during the surgical intervention. Only the tip of a surgical instrument reaches a field of view of the surgical microscope if it is positioned appropriately.

However, a view of a surrounding area of the microscope picture is extremely important in order to avoid damage outside the actual intervention area. In order to see the surrounding area, the surgeon has to look away from the binocular system or the display device of the microscope, quickly visually capture the surroundings of the area targeted by the surgical microscope and then turn back to the microscope picture, which leads to enormous strain during an intervention. A constant change between different viewing directions on the two viewing modalities as well as a constant correlation in the surgeon's head are tiring and often lead to unintentional errors.

The surgical microscope also has to be arranged in an operating room in such a way that the surgeon in charge, in particular, can easily handle and look into the surgical microscope or has a good field of view of an OR monitor on the one hand, and has a good view of the operating field/operating area/intervention area on the other hand. Thus, the OR monitor has to be arranged such that the surgeon has a good view of both modalities, but this limits flexibility of an arrangement in the operating room. It is also difficult for the surgeon to have a direct view of the intervention area, as the installation volume of a microscope head of the microscope usually blocks the direct view of the patient. In addition, this position and correspondingly good view is reserved only for the surgeon, whereas other surgical staff in other positions in the operating room have a difficult view of the microscope picture displayed by the OR monitor on the one hand and of the intervention area on the other hand. Medical staff is thus forced to constantly turn their heads and change the direction of view if the position is unfavorable.

For example, US 20190290101 A1 discloses an assistance system comprising a surgical microscope used in combination with a portable visualization system provided on a surgeon's head. Through this visualization system, the surgeon is enabled to see either a microscope picture or only a kind of magnifying image/magnification view, depending on the viewing angle of his eye. This allows the surgeon to switch between two modalities, but such a visualization system is associated with limitations and difficulties. Due to the integrated optics, the visualization system is heavy, expensive to produce and error-prone, since a beam path to the surgeon's eye must always be correct. The surgeon also loses perception of the surroundings due to the visualization system.

U.S. Pat. No. 9,936,863 B2, for example, discloses a system with a surgical microscope that has additional cameras on a wound spreader device on the patient in order to provide the surgeon in particular with a central microscopic image of the surgical microscope as well as peripheral images of the additional cameras provided on the wound spreader device. However, such a system has the disadvantage that, on the one hand, a wound spreader with cameras has to be provided on the patient and, on the other hand, an environment around the intervention site is not optically captured, which, however, provides the surgeon with crucial information.

### SUMMARY

It is therefore the object of the present disclosure to avoid or at least reduce the disadvantages of the prior art and in particular to provide a surgical assistance system, an (image) display method, a storage medium as well as a sterile space, which allows operating room participants/medical staff, in particular a (leading) surgeon, to view a central intuitive and supportive fusion of information of a (magnified) microscope image/microscope picture of a targeted area of an intervention site as well as a view of an environment around the targeted area of the intervention site. In addition, hand-eye coordination shall be improved and safe access to the intervention area/operation area/operation field shall be ensured, in particular to avoid or at least minimize tissue damage due to careless and unintentional actions as well as a duration of a surgical intervention. In particular, the surgeon's head movement necessary for the intervention as well as mental stress should be minimized by linking information from the microscope image and the environment.

Basically, the surgical microscope, in particular the microscope head, is equipped with a camera system with at least one additional camera. The camera is arranged on the microscope head, in particular on the housing of the optical microscope system, in particular fixed/rigidly attached. Thus, the optical microscope axis points in a similar viewing direction as the optical camera axis. Thus both images, the microscope image and the camera image are in particular correlated. The additional surrounding(s)/environmental camera generates images with lower magnification compared to the surgical microscope or preferably wide-angle images/wide-angle pictures with similar viewing direction as the optical system of the surgical microscope. The surrounding(s) camera or an additional camera of the camera system can also be arranged on the microscope head in such a way that it is arranged or integrated within the microscope head, in particular within the housing.

Both images, the microscope image by the surgical microscope (or the optical system) and the surroundings image by the surroundings camera are processed by the control unit and a combined view with both images is generated. This (digitally generated) combined view, in which information on both images is visually available, is then centrally output by the display device.

The assistance system thus enables the user, in particular the surgeon, to visually grasp both the interior of the intervention area in enlarged form and the surroundings/exterior of the intervention area for an overview, without having to change his head position or his viewing direction. In particular, the central view improves coordination, counteracts fatigue and shortens an intervention. It also increases a patient's safety, since a better overview is provided to the surgeon.

In other words, the surgical microscope, in particular the microscope head, comprises a camera system with at least one surroundings camera, wherein the surroundings camera is arranged on or connected to the microscope head. In particular, the surroundings camera is attached to the housing. Preferably, the surroundings camera is fixed rigidly to the microscope head, in particular to the housing, so that an orientation relative to the microscope head and the optical system always remains constant. The surroundings camera is adapted in such a way, in particular via an orientation of its optical camera axis and/or via its camera optics, that a field of view of the surroundings camera, in particular from a predetermined distance, preferably from a distance of 5 cm to a front of the camera or to a side of the microscope head, includes/encompasses/comprises/involves the field of view of the optical system of the surgical microscope in order to detect, by the surroundings camera, both the area targeted by the optical system and surroundings around the targeted area and to provide a surroundings image digitally/computer-readable. The control device is further adapted to process the surroundings image and to generate a combined view with the microscope image and the surroundings image and to output it visually by the display device.

In this case, a combined view means that the information of both the microscope image and of the surroundings image are centrally displayed in a single view—the combined view. In other words, the combined view shows the microscope image and the surroundings image, processed by image processing if necessary.

In particular, the surroundings camera has a similar or parallel viewing direction to the optical system of the surgical microscope. Thus, in addition to the targeted area, an environment around this targeted area is also optically recorded. It can also be said that the optical axes of the optical system and the surroundings camera are aligned similarly, in particular parallel or substantially parallel to each other. At this point it is noted that an optical detection in particular does not have to be limited to the visible area of the light, but may also include other frequencies below and above the visible area. For example, an infrared image can also be recorded by the surroundings camera as a surroundings image, can be processed by the control unit and can be output accordingly. Preferably, the surroundings camera, in particular the surroundings camera and the control unit, or another camera of the camera system can be adapted to record or generate a 3D picture and/or a so-called depth map. In this way, not only a two-dimensional picture is obtained, but also a 3D structure of the surroundings. A depth map contains information about distances from a viewpoint to a surface, so that a 3D structure is detected.

The term field of view defines an area in the picture angle of the optical system or of the surroundings camera, within which an image can be taken. It is, so to speak, the picture/image that falls on the image unit or a sensor, limited by the edges. This can be compared to a cone, which is coaxial to the optical axis and whose tip is located at a front of the optics. All ‘objects’ in the inner volume of the cone can be recorded. An aperture angle of the cone defines the possible image area. In this case, the cone of the microscope is graphically located in the cone (inside) of the surroundings camera. Thus, the surroundings camera can detect the targeted area on the one hand, since the targeted area is also inside the cone of the surroundings camera, and on the other hand an environment around this area. While the surroundings camera has a wide aperture angle (of the cone) with correspondingly small magnification, the optical system of the microscope has a very narrow aperture angle with correspondingly high magnification.

Advantageous embodiments are claimed in the dependent claims and are explained below.

According to a preferred embodiment, the assistance system may further comprise a storage unit with preoperative 3D image data stored therein, in particular MRI image data and/or CT image data, and the assistance system, in particular the control unit, can be adapted to spatially detect a (three-dimensional) 3D structure of the patient, in particular of a head, in particular preferably of a face of the patient, via the camera system, in particular with only the surroundings camera, and to correlate the detected 3D structure with the 3D image data in order to register the patient, in particular with respect to the surgical microscope, in particular preferably the microscope head. In other words, in particular the control unit may be adapted to correlate a recorded 3D structure with the 3D image data based on stored 3D image data of the patient, in particular stored data of a magnetic resonance imaging (MRI) or computed tomography (CT), in order to register the patient. In this way, a local coordinate system of the patient is linked to the local coordinate system of the surgical microscope, in particular of the microscope head, via a transformation matrix. In this way, the 3D image data can be geometrically related to the patient or linked to the microscope head. The patient can thus be registered in a simple manner via the surroundings camera. If the microscope head is moved, the movement of the microscope head can be detected/tracked and can be correlated with the 3D image data accordingly. Thus, another function of the assistance system with the surroundings camera is to register the patient when using a surgical navigation system. In yet other words, the additional surroundings camera can be used to register the patient by pattern recognition. The 3D surface of the patient, for example the face, is extracted and correlated with the 3D surface of 3D image data/of a preoperative 3D dataset, such as CT image data or MRI image data. The surroundings camera thus generates a 3D picture of a patient surface, for example of the face, which can be correlated with preoperative 3D image data, for example MRI image data, for which the control unit is adapted accordingly. External references for correlation may be in particular rigid bodies and/or optical patterns and/or characteristic landmarks. The surroundings camera is aligned to the patient on the area that is used for registration, for example the face. The term 3D defines that the structures, image data or surfaces of the patient are spatial, i.e. three-dimensional. The patient's body or at least a part of the body with spatial dimensions can be digitally available as image data in a three-dimensional space with, for example, a Cartesian coordinate system (X, Y, Z).

According to a further embodiment, registration via at least one marker attached to the patient can be performed in addition or as an alternative to registration via the 3D structure. The control unit is adapted to register the patient with respect to the surgical microscope, in particular the microscope head, via a detected predefined position of the at least one marker. The registration can thus be performed with markers or without markers (markerless) using an image processing technology. The term pose defines both a position and an orientation.

Preferably, for the detection of a 3D structure, the camera system may have a 3D camera and/or a 2D camera, in particular consisting of only the surroundings camera as the only 2D camera or 3D camera of the camera system. In the case of the 2D camera, it can be moved over an area of interest of the patient in order to obtain different views of the area of interest. The control unit is then adapted to compute a 3D structure from the different captured views using image analysis (machine vision). Thus, in order to create a 3D surface/3D structure, a 3D camera is used or a 2D camera that is moved over the patient and generates pictures from different perspectives. The 3D structure/3D surface generated in this way can then be correlated via the control unit, in particular with preoperative 3D image data.

According to an aspect of the disclosure, the assistance system may in particular comprise at least one sensor and may be adapted to detect a position, in particular a pose, of the microscope head and to provide it to the control unit as position data or pose data. The control unit may preferably be adapted to store the position, in particular the pose, of the microscope head, upon registration, in a storage unit as a registration position and an associated surroundings image, in particular a detected 3D structure. Upon re-registration in the stored registration position or pose, the control unit can determine that a registration deviation exists if an overlay of the stored 3D structure and the re-detected 3D structure deviates beyond a predetermined threshold, in particular in partial areas of the overlay that do not change during an intervention (such as covers or wound edges/incision borders). In particular, the control unit may be adapted to calculate an amount of deviation. For example, a set of characteristically predefined points can be determined and a distance between the corresponding points of the stored 3D structure and the newly detected 3D structure can be summed up. If the sum of the distances exceeds a threshold value, it can be determined that a deviation exists. Preferably, the control unit may be adapted to automatically correct the registration by an offset, in particular by an offset of a translation in space and/or a rotation about an axis. In other words, the assistance system may preferably be adapted to detect a deviation between two surroundings images (from the same pose), calculate an offset/displacement/deviation, and correct for the predetermined offset. In particular, the control unit may inform a user in the presence of a deviation, for example visually via the display device and/or acoustically via an audio device. In particular, an alarm signal can be output via the assistance system. In particular, the assistance system can use the surroundings camera and the adapted control unit to automatically detect and correct inaccuracies in navigation caused, for example, by unintentional movements of a patient tracker. The surroundings camera and the optical system of the surgical microscope are geometrically correlated. During or after initial registration, the surroundings camera captures a picture of the intervention area from a specific position (registration position) or pose. This position or pose is calculated as a relative transformation (transformation matrix) of a patient tracker to a microscope tracker. In order to check the accuracy, the microscope head can be moved (back) to this registration position at any time during the operation to capture a picture (surroundings image) again. Both pictures—the one from the original registration and the one from the accuracy check (the renewed surroundings image) are superimposed. If there is no inaccuracy or deviation, the pictures match in all special picture sections/picture areas that are not changed during the intervention (such as covers or wound borders/incision borders). If, on the other hand, there is a deviation or inaccuracy, the two images/pictures are offset from each other. In particular, the user can be informed about the deviation. Furthermore, by calculating an offset, the surgeon can also be informed about the amount of the deviation. In addition, the inaccuracy can preferably be corrected automatically by the control unit by adjusting the registration with the determined offset.

According to a further aspect of the disclosure, alternatively or in addition to the registration position to be stored, a geometric position, in particular pose of the microscope head, can also be predetermined, into which the microscope head is moved in order to perform the surroundings image from there, in particular via the detection of an intervention area or a detection of a face of the patient. The above features of the configuration of the assistance system for calculating the deviation are also found in this aspect.

In particular, the surgical microscope may comprise at least one actuator in order to actively move and control the movable microscope arm and the movable microscope head, and the control device may be further adapted to control the actuator to move the microscope head to a predetermined position and/or orientation in space. In this way, the microscope head can be ‘moved’ automatically, without manual handling, in particular to a predetermined position (position and orientation). In this way, for example, the registration position explained above can be assumed with unambiguous repeat accuracy, or a stored view can be ‘recalled’ by the surgical microscope.

According to an aspect of the invention that may be claimed independently, the surroundings camera and/or the image unit may comprise a high-resolution sensor preferably having at least 35 megapixels, and the control unit may be adapted to digitally magnify one, in particular central, area of the sensor for an image, while using a lower magnification for another area of the sensor.

According to a further aspect of the disclosure, the control unit can be adapted, in particular initially, to create a surroundings image, to extract at least one characteristic landmark in the surroundings image, and to determine a position, in particular a pose, of the extracted landmark relative to the microscope head and thus of the landmark in space with respect to a reference coordinate system on the basis of a detected pose of the microscope head (with defined geometric relation of microscope head to surroundings camera), and to store the characteristic landmark of the surroundings image and the associated determined position or pose as a reference value in the storage unit, wherein the control unit is further adapted to continuously recognize this extracted landmark in a current surroundings image and to continuously determine a current position, in particular a current pose, of the landmark in space and, in the event of a deviation between the stored position or pose and the current position or pose, to determine that there is a deviation in the registration. In particular, the control unit then outputs a corresponding message via the display device. In other words, in one embodiment, instead of detecting deviations in a registration position, a method or a correspondingly adapted control unit can also be used to determine a deviation of the registration in positions or poses that are not predetermined. In this case, a partial area of the area observed or detected by the surroundings camera, which does not change during the intervention, is used. In this area, traceable landmarks are extracted using image processing methods. Landmarks can be found or determined initially at the beginning of the intervention or procedure, or continuously during the intervention. The landmarks have to be of such a nature that they are detectable under different positions and/or orientations of the microscope head by means of the surroundings camera. The position of each landmark lying in the field of view of the surroundings camera is continuously determined relative to the surroundings camera, in particular by means of image processing methods. Due to the known geometric relation or pose of the surroundings camera relative to the microscope head and due to a measurable position of the microscope head, the position of the observed landmarks is known and can be calculated or determined by the control unit. The landmark position determined in this way can then be compared with the expected landmark position or pose (target position). If a deviation is observed here, a deviation of the registration can be assumed. The expected landmark position is stored in the system or in the storage unit, in particular initially during the first observation or detection of a landmark, in order to be able to use it as a reference value for further detections of the landmark.

According to a further aspect of the disclosure, the control unit may be adapted to determine a position, in particular a pose, of a landmark relative to the surroundings camera by means of image processing methods and to compare it with a known pose of the landmark, in particular with a detected pose of the landmark by an external camera of the navigation system and/or a detected pose by the optical system and the image unit of the microscope, and to improve a registration and/or minimize an error in camera systems using minimization methods of the overdetermined system. In other words, according to one embodiment, the determined position or pose of the observed or characteristic landmarks relative to the surroundings camera can also be used to improve an accuracy of the navigation system or a registration. For this purpose, analogously the assumption is made that the pose of the microscope head, the pose of the surroundings camera and the pose of the landmarks are known or can be determined. If the position, in particular the pose, of the landmarks is determined (by the control unit) via image processing methods relative to the surroundings camera, there is another way to determine the position of the landmarks via the position, in pustular the pose, of the microscope or microscope head. Due to inaccuracies in camera systems or registration, certain landmark positions will differ between the two ways. From a mathematical point of view, this results in an overdetermined system. Using minimization techniques, this overdetermination can be used to continuously improve errors in camera systems and/or in a registration of the navigation system, thus increasing the accuracy of the overall system.

According to a further aspect of the disclosure, in the case of an actively movable microscope head, the assistance system may comprise an input unit, in particular a touch screen, for selecting a focus point in the surroundings image of the surroundings camera. The control unit may in turn be adapted to actively control and move the microscope arm and the microscope head based on the selected focus point via the at least one actuator such that the optical microscope axis is aligned with the selected focus point at the patient, in particular at a predetermined associated distance and/or at a predetermined image angle. Thus, an intuitive, automatically adjustable surgical microscope is provided that allows the surgeon to align the microscope for appropriate focus with just one touch on a selected point in the surroundings image. Thus, an additional function of the assistance system with the surroundings camera is to move a robot-guided surgical microscope into a target field by defining a target/focus point in the surroundings image of the surroundings camera and moving the microscope such that the focus point is in the focus of the microscope image or of the optical system of the surgical microscope.

According to a further aspect of the disclosure, the control unit may be adapted to detect, via image analysis of the surroundings image and/or a sensor provided on the microscope head, an object in the area of the microscope head as well as a distance of the movable microscope head to this object. In the event of a possible collision with this object, in particular if the distance falls below a predetermined distance, the assistance system, in particular the control unit, is adapted to emit an alarm signal, in particular an alarm sound via an audio device and/or an alarm display via the display device and/or to restrict at least a degree of freedom of movement of the microscope head and/or of the microscope arm, in particular to stop overall movement of the microscope head, in particular of the entire surgical microscope. A further function of the assistance system with the additional surroundings camera is thus to prevent collisions of the microscope with objects by the assistance system determining a distance to the microscope head and issuing a warning visually or acoustically if the distance is undershot or, in the case of a robot-guided surgical microscope, stopping the movement. The surroundings camera/overview camera can also detect collisions during autonomous movement of the microscope head, which is guided over the microscope arm, in particular in the form of a robot arm. The movement can then be stopped in time, for example by a braking or locking system. Even if the microscope arm or robot arm is guided manually, a collision can be detected and further movement can be prevented.

Preferably, the control unit can be adapted to use the camera system, in particular at least the surroundings image, in particular markers and/or characteristic features of the patient, to correctly detect and in particular track the pose of a medical, in particular surgical, instrument and/or the microscope head. The surroundings camera can therefore continuously detect in particular the pose of a medical instrument. This pose of the detected medical instrument can in particular be reproduced in the 3D image data by schematically displaying a virtual position and/or orientation or by superimposing a virtual instrument in the corresponding position. In particular, a relative pose of the patient and/or medical instruments with respect to the microscope head can be displayed correctly in the 3D image data. Furthermore, in particular, a detected microscope head can also be displayed in the 3D image data as a virtual microscope head. In other words, the assistance system with the surroundings camera can be used in particular for tracking a position, in particular a pose, of the patient when using the surgical microscope. Preferably, external references, such as rigid bodies or optical patterns or unique anatomical landmarks, can be used for this purpose to determine or locate the position, in particular pose, of the patient relative to the microscope head. This allows to display the microscope picture correlated with the preoperative 3D image data, such as MRI image data. Preferably, the surroundings camera can also be used to track surgical instruments, in particular those equipped with markers, to superimpose their position, in particular pose, in the microscope picture of the preoperative 3D image data. Thus, another function of the assistance system with the additional surroundings camera is to provide it for tracking/tracing the patient and/or a surgical instrument and/or the surgical microscope itself when using a surgical navigation system. For example, markers in the field of view of the surroundings camera can be used for tracking, or tracking can be performed without markers using unique characteristics of the patient or the instruments. In this case, the surroundings camera can replace the external camera typically used in surgical navigation systems. In addition, the surroundings camera can also temporarily replace or supplement an external camera of the surgical navigation system in order to detect and, in particular, localize features and instruments even in the event of temporarily occurring visibility restrictions (for example, due to overlapping) of the external camera.

According to a further aspect of the disclosure, data, in particular geometric relations, of at least one medical instrument and associated use instructions may be deposited/stored in a storage unit. The control unit may further be adapted to detect a surgical instrument in the surroundings image based on the stored data, in particular based on the stored geometric relation, and to output the associated stored use instructions to a user via the display device and/or an audio device. An additional function of the assistance system with surroundings camera is thus the recognition of used medical, in particular surgical, instruments as well as a provision of associated use instructions for the surgeon.

According to an embodiment, the at least one display device may be an OR monitor and/or a head-mounted display (HMD) and/or a binocular system with data capable of fading in. In case of the OR monitor as display device, the combined view is displayed as a picture on the OR monitor. In the case that the assistance system has a head-mounted display/virtual reality goggles as display device, the combined view can be output via a display. In particular, if the surgical microscope and/or the surroundings camera is adapted to create a 3D image, the combined view can be output three-dimensionally via the head-mounted display so that a spatial view is provided to the surgeon. In the case of a binocular system, through which the surgeon looks with both eyes, the surrounding data can be superimposed in a picture plane, for example via a prism and a display. This constitutes an augmented reality system. In other words, the at least one display device can be an (OR) monitor and/or a head-mounted display and/or a binocular system.

In particular, the control unit can be adapted to create a side-by-side or overlaid display of the microscope image and the surroundings image as a combined view and output it via the display device. In other words, the microscope image and the surroundings image can be displayed side by side or superimposed. In the case of a superimposed view, the microscope image (e.g. without transparency) can be created by the control unit in a central area of the combined view, whereby the surroundings image is displayed as a background around this microscope image.

According to a further embodiment, a magnification of the optical system can be at least fivefold, in particular at least tenfold, particularly preferably at least fortyfold. Preferably, the optical system of the surgical microscope can have a zoom function. Further preferably, a magnification of the surroundings camera can be at most fivefold, in particular at most single. While the surgical microscope with the optical system and the image unit provides a relatively high magnification, the surroundings camera with its optics is adapted to create a wide-angle image and provide it as a surroundings image for a view of the environment.

According to one aspect of the disclosure, the camera system may further comprise, in addition to the surroundings camera, a second camera, in particular, in addition, a third camera, particularly preferably, in addition, a fourth camera, in order to enlarge a field of view around the intervention area. Thus, in the camera system, in addition to the surroundings camera, additional cameras may be used to enlarge the field of view outside the operation field.

In particular, a focal length of optics of the surroundings camera may be a minimum of 10 mm and/or a maximum of 50 mm or a viewing angle may be at least 45°, preferably at least 70°, to provide a wide angle surroundings image.

Preferably, the optical microscope axis can intersect the optical camera axis or pass it at a distance of at most 10 cm, particularly preferably at most 5 cm. In particular, an angle between the optical microscope axis and the optical camera axis can be a minimum of 2° and/or a maximum of 15°. Alternatively, the optical microscope axis and the optical camera axis are preferably parallel to each other.

With respect to an (image) display method for displaying two different images, the objects and aims of the present disclosure are solved by the steps of: preferably arranging a surroundings camera on a movable microscope head of a surgical microscope; targeting an area via an optical system arranged on the movable microscope head; creating a microscope image by a microscope image unit arranged on or in the microscope head of a magnification provided by the optical system; creating a surroundings image by a surroundings camera arranged on the microscope head, wherein a field of view of the surroundings camera, in particular from a predetermined distance, includes a field of view of the optical system; creating a combined view with the microscope image and the surroundings image; outputting the combined view by a display device. The display method provides the surgeon with both image modalities centrally.

The display method may preferably further comprise the steps of: reading in preoperative 3D image data, in particular MRI and/or CT image data; detecting a 3D structure of a patient by the surroundings camera; correlating the detected 3D structure with the 3D image data; and registering the patient via the correlation.

According to an embodiment, the display method may comprise the steps of: initially registering the patient; detecting a pose of the microscope head; creating a surroundings image by the surroundings camera; storing the detected pose (registration position) and surroundings image; moving the microscope head and then returning to the stored pose; renewing the surroundings image; comparing the stored surroundings image with the renewed surroundings image; determining a deviation between the stored and the renewed surroundings images; outputting an acoustic or visual signal if the deviation exceeds a threshold value and/or calculating an offset and applying the offset to the initial registration.

In particular, as described above for the surgical assistance system and applicable analogously to the method, a position of the observed landmarks can also be continuously compared with a reference position of the landmarks and any deviation can be determined. If a deviation is detected, it can be determined in particular that a deviation of a registration exists and preferably a message may be issued. When using a method with continuous detection of a registration deviation, storing the registration position, imaging the environment in the registration position and returning to the registration position with imaging of the environment can be omitted.

According to a further embodiment, the display method may comprise the steps of: detecting a medical instrument and its position and/or orientation in the surroundings image; transferring the pose of the medical instrument into 3D image data and view of a virtual medical instrument or at least a schematic view in the 3D image data;

Preferably, the display method may comprise the steps of:
tracking of a patient and/or a surgical instrument and/or the microscope head by the surroundings camera and provision of the corresponding movement data.

Furthermore, the display method may in particular comprise the steps of: determining a distance to an object detected in the surroundings image; if the distance is undershot: outputting an alarm signal and/or restricting degrees of freedom of movement of the microscope head, in particular stopping/blocking a movement of the microscope head. The surroundings image of the surroundings camera/overview camera can also be used to detect collisions during autonomous or collaborative movement of the microscope arm, in particular in the form of a robot arm. The movement can then be stopped in time by a braking system. Even if the microscope arm or robotic arm is manually guided by the surgeon, for example, a collision can be detected and further movement can be prevented.

According to one aspect of the disclosure, the display method may comprise the steps of: reading a focus point in the surroundings image; controlling a microscope arm and the microscope head such that the optical microscope axis is aligned with the selected focus point, in particular at a predetermined distance and/or at a predetermined image angle.

Preferably, the display method may comprise the steps of: detecting a medical instrument in the surroundings image, outputting use instructions associated with the medical instrument via the display device.

At this point, it is noted that features of the display method of the present disclosure are transferable to both the surgical assistance system of the present disclosure and vice versa.

With respect to a computer-readable storage medium, the objects and aims of the present disclosure are solved in that the computer-readable storage medium comprises instructions which, when executed by a computer, cause the computer to execute the method steps of the image display method according to the present disclosure.

With respect to a generic sterile space, the object of the present disclosure is solved in that the medical sterile space comprises a surgical assistance system according to the present disclosure.

### BRIEF DESCRIPTION OF THE DRAWINGS

The present disclosure is explained below with reference to preferred embodiments with the aid of accompanying figures.

FIG. 1 shows a schematic perspective view of a surgical assistance system of a first preferred embodiment, in which the surgical assistance system is provided as a mobile surgical microscope.

FIG. 2 shows a schematic perspective view of a microscope head of the surgical microscope of FIG. 1.

FIG. 3 shows a further schematic perspective view of the assistance system of FIGS. 1 and 2 during an intervention.

FIG. 4 shows a schematic view of a surgical assistance system according to a further preferred embodiment with a head-mounted display.

FIG. 5 shows a schematic view of a detection of a 3D structure by the surroundings camera.

FIG. 6 shows a flowchart of an image display method according to a preferred embodiment.

The figures are merely schematic in nature and are only intended to aid understanding of the disclosure. Identical elements are provided with the same reference signs. The features of the various embodiments can be interchanged.

### DETAILED DESCRIPTION

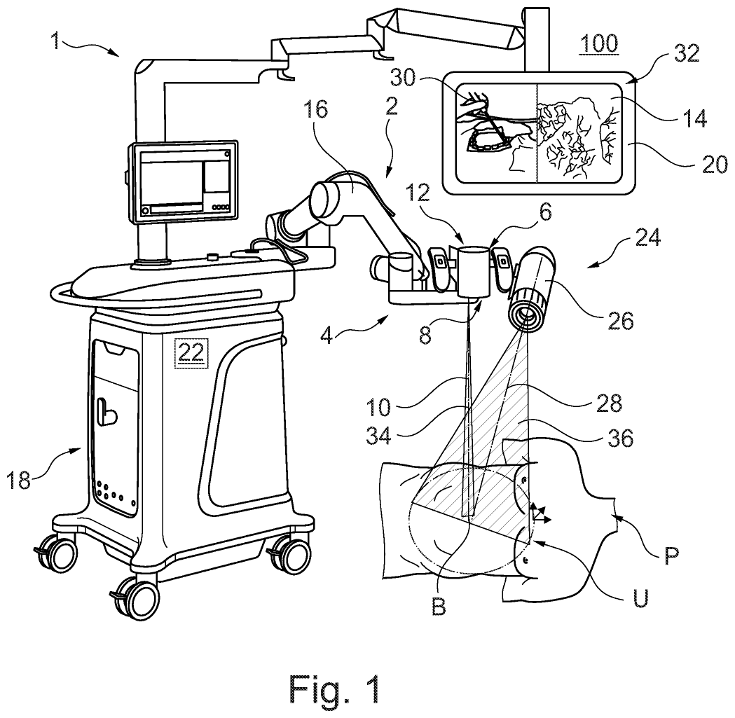

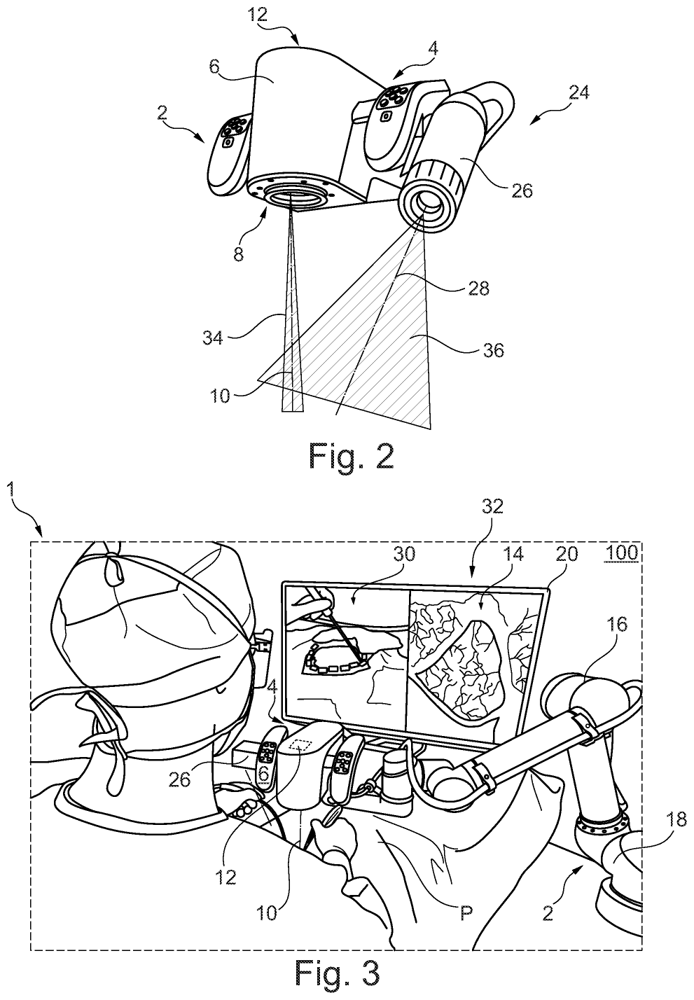

FIGS. 1 to 3 each show a schematic perspective view of a surgical assistance system 1 (hereinafter referred to as assistance system) of a first preferred embodiment for use in a surgical intervention on a patient P. The assistance system 1 is used in a medical sterile space in the form of an operation room 100 of a preferred embodiment in order to support operating room participants, in particular a surgeon, via suitable visualization during a surgical intervention on a patient P (shown here only schematically). In a central, sterile, surgical intervention area, a minimally invasive intervention is performed on the patient P.

In this preferred embodiment, the surgical assistance system 1 features a surgical microscope 2 and is even configured as a mobile, autonomously operating surgical microscope 2. Specifically, the surgical microscope 2 (hereinafter referred to only as microscope) has a front movable/adjustable microscope head 4 that is movably mounted. In this way, the position and orientation of the microscope head 4 can be individually adjusted.

The movable microscope head 4 has a housing 6 and an optical system 8 arranged in this housing with a plurality of optical elements such as lenses or lens systems. In this embodiment, a monocular optical system is provided. However, a binocular optical system 8 can of course also be provided, for example to create 3D microscope images. The optical system is adapted to provide an optical magnification of a targeted area B, as it is common in surgical microscopes, in the direction of an optical microscope axis 10. In particular, the optical system is adapted to sharply focus and provide a magnification of the targeted area B located opposite a front lens (not shown) at a distance between 5 cm to 80 cm. This provided magnification is detected by a downstream microscope image unit 12 (hereinafter referred to as image unit) comprising a sensor, such as a CMOS sensor or a CCD sensor, and the image unit produces a digital/computer readable microscope image 14.

The microscope head 4 is also mounted on a movable microscope arm 16 supported by a base 18 in the form of a trolley. The microscope arm 16 in the form of a, in particular collaborative, robotic arm with two arm elements with corresponding mounting of the base 18 and of the microscope head 4 enables the microscope 2 to set a position and/or orientation of the microscope head 4 in space and to align it with an area B to be targeted, which the surgeon wants to see magnified.

In order to display this magnification visually, the assistance system 1 or the mobile microscope 2 has an OR monitor 20 on a cantilever. A control unit 22 processes the microscope image 14 and outputs the view via the OR monitor 20.

In contrast to known assistance systems, the microscope head 4 of the microscope 2 additionally has a camera system 24 with a surroundings camera 26 (hereinafter referred to as camera). This camera 26 is rigidly attached to the housing 6 of the microscope head 4 so that the optical system 8 of the microscope 2 and the camera 26 always have the same relative orientation to each other. Specifically, the surroundings camera 26 is adapted via an orientation of its optical camera axis 28 and via its camera optics for a (camera) field of view 36 of the surroundings camera 26 to include a (microscope) field of view 34 of the optical system 8 from a predetermined distance in order to detect by the surroundings camera 26 both the area B targeted by the optical system 8 and surroundings U around the targeted area B and to provide a corresponding surroundings image 30 by the camera 26.

The control device 22 is adapted to process this surroundings image 30 and to generate a combined view 32 in the form of a side-by-side display with the microscope image 14 and the surroundings image 30 and to output it visually by the display device 20.

The movable microscope head 4 is therefore equipped with an additional surroundings camera 26. The viewing direction or the orientation of the optical camera axis 28 of the camera 26 is similar to the viewing direction or the optical microscope axis 10 of the optical system 8 of the microscope 2. This additional camera 26 generates a wide-angle view, which makes it possible to capture the surroundings U of the operation field around an area B targeted by the optical system 8, in particular to capture incision borders, the surgeon's hands or instruments used. The view of the picture/image of the camera 26 together with the microscope image allows the surgeon to see both ‘the inside’ and ‘the outside’ of the intervention area without changing his head position or viewing direction. In this regard, the camera 26 is mounted in fixed relation to the microscope head 4 and is adapted to image or detect the surroundings U of the intervention area even at close distances between the microscope head 4 and the patient P of, for example, 10-30 cm. With the assistance system 1, the surgeon sees both the microscope image 14 with high magnification and the additional surroundings image 30 with low magnification on the OR monitor 20. Thus, the surgeon can intuitively see both an inner high-resolution area and a clear outer area of the intervention area with one glance.

The microscope head 4 thus has two different ‘optical systems’, namely the optical system 8 of the microscope 2 and an optical system of the camera 26, which are correlated to each other by their arrangement. The optical system 8 of the microscope 2 serves to generate a picture with high magnification and the additional camera 26 with associated optics serves to generate a picture with wide angle. Both optics are correlated and the viewing directions are similar and can be directed to the intervention area/operation field. The camera system 24 with the camera 26 has a low magnification and wide field of view 36 to detect an operation environment with, e.g., an incision border or hand and/or instrument around the intervention site, while the optical system 8 has a high magnification with lower field of view 34. The combined view 32 then displays the two images 14, 30 centrally and allows the surgeon as well as other medical personnel to see both image modalities correlated to each other at a glance.

In addition to the camera 26, the camera system 24 may also have a further camera attached to the microscope head, for example to create a stereo image or to capture multiple views and combine them accordingly in the combined view 32. Also, the camera 26 itself may be a 3D camera or stereo camera in addition to a 2D camera.

The camera 26 has an autofocus function to focus on the targeted area B or the surroundings. Moreover, the camera 26 may have a zoom function to optimally adjust a surroundings image 30 to a particular intervention. Additionally, the optical system 8 may also have a zoom function to adjust a magnification of the microscope 2. The zoom function may be continuous via lenses that move relative to each other as well as discrete in the manner of a revolving nosepiece. In particular, the surgeon can adjust a zoom/magnification via a knob on the microscope head. Also, the camera 26 and/or optical system 8 may have an adjustable iris to control a focus and light input.

Since the control unit 22 digitally processes the two images, it can perform various digital analyses, image processing and controls, in particular to generate a suitable combined view 32. For example, the control unit 22 can be adapted to change various picture parameters, such as brightness or contrast in the images. Further digital data can also be included in the combined view 32. The control unit 22 may comprise a central processing unit (CPU), a volatile memory such as RAM, ROM and a non-volatile memory such as SSD.

The microscope arm 16 is actively controllable via a number of actuators. Sensors provided in the individual elements of the microscope arm 16 enable a determination of the pose of the microscope arm 16 and of the microscope head 4, which is passed on to the control unit 22. The surgeon can manually select a focus point with a finger in the surroundings image 30 on the OR monitor 20, which is designed as a touchscreen. This set focal point is then passed on to the control unit 22, which in turn actively controls and moves the actuators of the microscope arm 16 and the bearing of the microscope head, and on the basis of the set focal point, the optical microscope axis 10 is aligned with the focal point on the patient P at a predetermined distance and at a perpendicular image angle. In this way, the targeted area B is set to the focal point and the surgeon can easily perform his intervention at this point or display a magnified image. The focal point can also be saved in order to switch automatically between different views.

Additionally, the control unit 22 is further adapted to detect an object via a picture analysis of the surroundings image 30 and further to calculate a distance to the object. Alternatively or additionally, distance sensors can also be provided on the microscope head 4, in particular several distance sensors in different directions, in order to determine an object and a distance. In the event of a possible collision with the object thus detected in the field of movement, the assistance system 1 emits an acoustic alarm signal (collision warning) via an audio device (not shown) and blocks a movement of the microscope arm 16. In particular, a movement of the microscope arm 16 and of the microscope head 4 is completely stopped (collision prevention). This prevents a collision with the object.

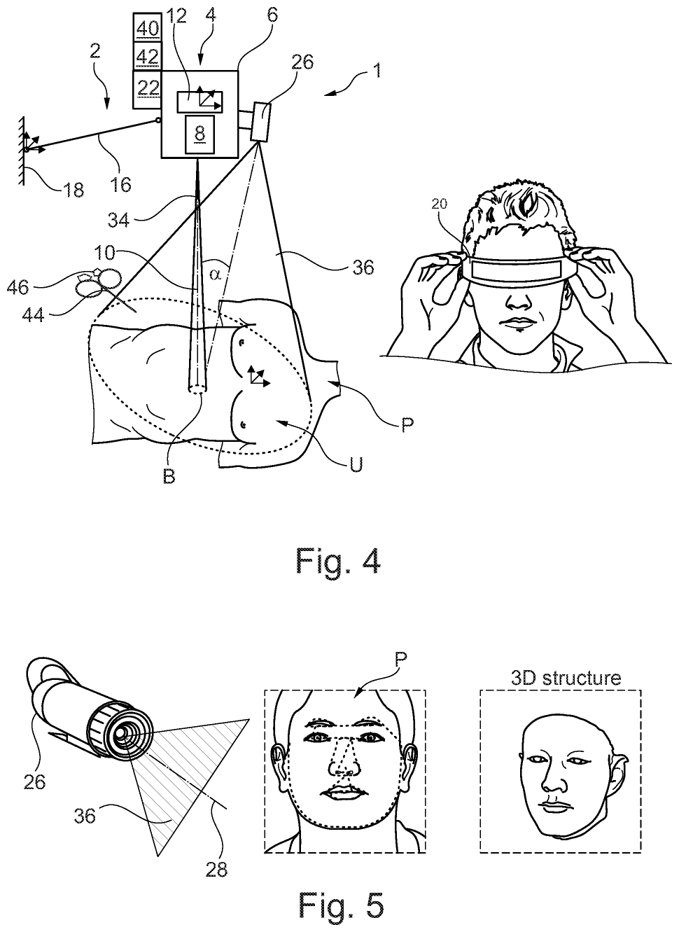

FIG. 4 shows a surgical assistance system 1 of a further, second preferred embodiment. Similar to the first embodiment, the microscope head 4 is movably mounted on a fixed base 18 via the microscope arm 16, and the optical system 8 is capable of targeting an area B. The surroundings camera 26 is oriented such that the optical camera axis 28 intersects the optical microscope axis 10 in the targeted area B. This ensures that the surroundings image is concentric or symmetrical about the targeted area. The control unit 22 again processes the two images 14, 30 and creates a combined view 32.

However, in contrast to that of the first embodiment, the combined view 32 is not output via a monitor, but via a head-mounted display/HMD glasses 38 (hereinafter referred to as glasses). For this purpose, the glasses 38 may comprise a display and additionally an optical system. Specifically, the control unit controls a transmitting and receiving unit 40, which transmits the data wirelessly to the glasses 38. There, the received data is then displayed in the glasses 38.

The assistance system 1 also uses a surgical navigation system and also has a storage unit 42 in which an MRI and a CT image of the patient P are stored as 3D image data. The control unit 22 is adapted to detect a 3D structure of the patient's face (see FIG. 5) in three dimensions when the microscope head 4 is correspondingly aligned with the camera 26. The 3D structure of the patient's face thus detected is matched with the virtual 3D structure of the patient's face of the 3D image data, and the two 3D structures are correlated to perform an initial registration and to register the patient P with respect to the microscope head 4. When the microscope head 4 moves, sensors detect a corresponding movement.

FIG. 5 schematically shows such a function of a registration over 3D structures of the patient's face as performed in the assistance system in FIG. 4. The camera 26 is a 2D camera that is moved over the area of interest of the patient's face (see dashed lines) to obtain different views of the face and to calculate a 3D structure of the face via machine vision methods or an image analysis performed by the control unit 22. The 3D structure of the face obtained in this way can be superimposed or correlated, as it were, with the virtual 3D structure of the 3D image data, or the control unit 22 can itself determine a position of the patient P based on the 3D structure.

Furthermore, the control unit 22 of the assistance system 1 in FIG. 4 is adapted to detect such a medical instrument 44 via markers 46 attached to medical instruments 44 or characteristic features, in particular geometric features, via the surroundings image 30 and to determine its pose and/or function. In the case of characteristic features, a virtual geometric relation of the instrument 44 can be stored in the storage unit 42, which the control unit 22 may use for matching. The medical instrument 44 thus detected can then be overlaid on the 3D image data at the acquired location and can be used for surgical navigation. In particular, the control unit can additionally overlay data for use instructions in the combined view to provide further guidance to the surgeon in his intervention. In particular, the detection of the instruments 44 via the surroundings camera 30 can be used for the control unit 22 to optimally position and/or align the microscope head 4 via the robot-guided microscope arm 16 in order to obtain, for example, a good field of view on the targeted area B without, for example, an instrument 44 obscuring the field of view or being positioned and/or aligned in such a way that an instrument tip is still in the field of view.

The control unit 22 is also adapted to automatically detect a deviation of a navigation due to unwanted movements of e.g. a patient tracker/marker. For this purpose, the assistance system 1 is adapted to move the microscope head 4 to a predetermined position (registration position and orientation, hereinafter referred to as registration position) after an initial registration, and to create a surroundings image 30 via the camera 26, which is stored in the storage unit 42. The registration position is calculated as a relative transformation between a patient tracker (not shown) and a microscope head tracker (not shown). In order to ensure that there is no deviation, the surgeon can (let someone) move the microscope head 4 to the registration position from time to time during an operation and produce a new surroundings image 30. This is then superimposed on the surroundings image 30 stored in the storage unit 42 and compared. If there is no or a small tolerable deviation, then partial areas of the two surroundings images 30 that are not subject to change, such as incision borders or surgical drapes, continue to correlate with each other and the control unit 22 determines via a corresponding image analysis that there is no or a tolerable deviation. However, if partial areas (with actually unchanging structures) of the two surroundings images 30 deviate from each other by more than a tolerable value/limit, the control unit 22 detects this and outputs a message to the surgeon via the glasses 38 or a monitor. Furthermore, the control unit 22 calculates a degree of the deviation via appropriate calculation methods and outputs the quantity of the deviation to the surgeon. Thus, the surgeon can determine if this deviation is too excessive and take appropriate action. Alternatively or additionally, image processing algorithms for realizing continuous detection of a registration deviation or continuous improvement of an accuracy of the registration may be stored in the storage unit 42 of the control unit 22.

Additionally or alternatively, the control unit 22 may also be adapted to determine a displacement/offset between the stored surroundings image 30 and the renewed surroundings image 30 and to adjust this offset to the registration or to a transformation matrix between a local coordinate system, such as that of the microscope head or the surgical navigation system and the local coordinate system of the patient P accordingly, and to correct for this offset.

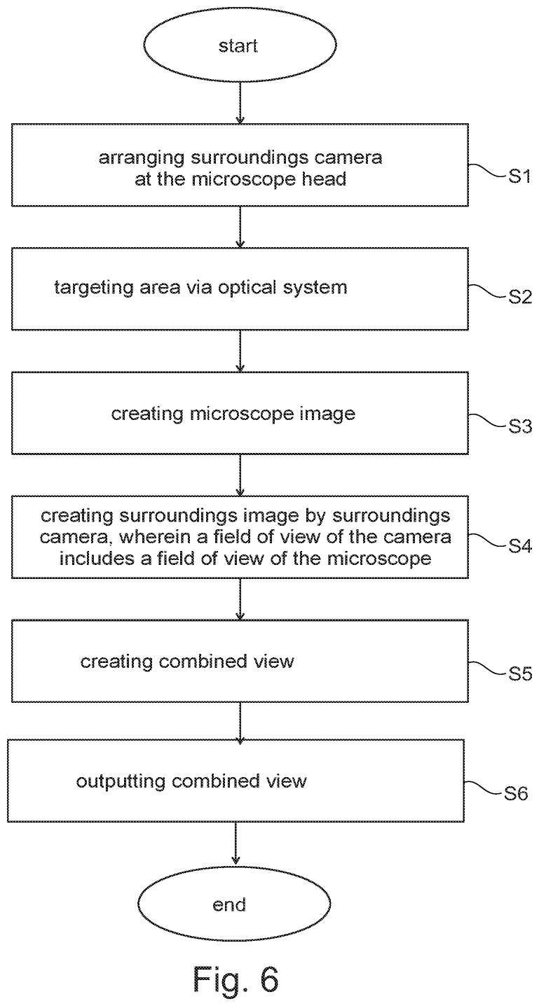

FIG. 6 shows a flowchart of an image display method (hereinafter referred to as display method) according to a preferred embodiment of the present disclosure for correlated display of a microscope image 14 and a surroundings image 30. The display method is used accordingly in a surgical assistance system 1 described above.

In a first step S1, a surroundings camera 26 can preferably be arranged on the movable microscope head 4 of the surgical microscope 2, in particular can be rigidly attached.

In a step S2, an area B is targeted by the optical system 8 arranged in the movable microscope head 4. In a subsequent step S3, the microscope image 14 is created by the magnification of the microscope image unit 12 arranged in the microscope head 4 provided by the optical system 8. In addition, in a step S4, a surroundings image 30 is created by the surroundings camera 26 arranged on the microscope head 4, wherein a field of view 36 of the surroundings camera 26 includes, in particular from a predetermined distance, a field of view 34 of the optical system 8.

In a step S5, a combined view 32 with the microscope image 14 and the surroundings image 30 is created by the control unit 22, and the combined view 32 is finally output by a display device 20; 38 in a step S6.

1.-14. (canceled)

15. A surgical assistance system for use in a surgical intervention on a patient, the surgical assistance system comprising:
A. a surgical microscope comprising:
i. a microscope head that is movable, the microscope head comprising a housing and an optical system adapted to provide optical magnification of a targeted area in a direction of an optical microscope axis and to create a microscope image via a downstream microscope image unit;
ii. a microscope arm that is movable and connected to a base, the microscope head being attached on the base, the base being adapted to adjust a position and/or orientation of the microscope head; and
iii. a camera system with at least one surrounding camera arranged on the microscope head, the at least one surrounding camera being adapted in such a way that a field of view of the at least one surrounding camera includes a field of view of the optical system to detect an area targeted by the optical system and surroundings around the targeted area and to provide a surroundings image;
B. at least one display device for displaying a visual content;
C. a control unit adapted to:
i. process the microscope image and control the at least one display device for a view accordingly, and
ii. process the surroundings image and generate a combined view with the microscope image and the surroundings image and output the combined view visually by the display device; and
D. a storage unit with preoperative 3D image data,
the control unit being adapted to spatially detect a 3D structure of the patient via the camera system and correlate the 3D structure with the preoperative 3D image data in order to register the patient.

16. The surgical assistance system according to claim 15, wherein the camera system comprises at least one of:
a 3D camera configured for detecting the 3D structure; and
a 2D camera configured to move over an area of interest of the patient in order to obtain a plurality of views of the area of interest, wherein the control unit is adapted to compute the 3D structure from the plurality of views using image analysis.

17. The surgical assistance system according to claim 15, wherein the surgical assistance system is configured to detect a position of the microscope head and to provide the position of the microscope head to the control unit, and the control unit is adapted to store the position of the microscope head in a storage unit as a registration position and an associated surroundings image as a first surroundings image, the assistance system further configured to renew the associated surroundings image as a second surroundings image, and upon renewing the associated surroundings image in the registration position, the control unit determines that a deviation exists when an overlay of the first surroundings image and the second surroundings image deviate beyond a predetermined threshold.

18. The surgical assistance system according to claim 17, wherein the surgical assistance system is configured to detect a pose of the microscope head and to provide the pose to the control unit, and the control unit is adapted to store the pose in the storage unit as a registration pose, and, upon renewing the associated surroundings image in the stored registration pose, the control unit determines that a deviation exists when a partial area overlay of the first surroundings image and the second surroundings image deviate beyond the predetermined threshold.

19. The surgical assistance system according to claim 15, wherein the surgical microscope comprises at least one actuator to actively move the microscope arm and the microscope head, and the control device is further adapted to control the actuator to actively move the microscope head to a predetermined position and/or orientation.

20. The surgical assistance system according to claim 19, wherein the surgical assistance system comprises an input unit for selecting a focus point in the surroundings image, and the control unit is adapted to actively control the microscope arm and the microscope head based on the focus point via the at least one actuator such that the optical microscope axis is aligned with the focus point at the patient at a predetermined associated distance and/or a predetermined image angle.

21. The surgical assistance system according to claim 15, wherein the control unit is adapted to:
detect an object in an area of the microscope head as well as a distance between the microscope head and the object via image analysis of the surroundings image and/or a sensor provided at the microscope head; and
emit an alarm signal and/or restrict at least a degree of freedom of movement of the microscope head when the distance falls below a predetermined distance that creates a risk of collision between the microscope head and the object.

22. The surgical assistance system according to claim 15, wherein the control unit is adapted to use at least the surroundings image of the camera system and to use markers and/or characteristic features of the patient to correctly detect and continuously track a pose of a medical instrument and/or the microscope head.

23. The surgical assistance system according to claim 15, wherein data including geometric relations, of at least one medical instrument and associated use instructions are stored in the storage unit, and the control unit is adapted to detect a surgical instrument in the surroundings image based on a geometric relation of the preoperative 3D image data and to output the associated use instructions to a user via the display device.

24. The surgical assistance system according to claim 15, wherein the at least one display device is an OR monitor and/or a head-mounted display and/or a binocular system with data capable of fading in.

25. The surgical assistance system according to claim 15, wherein a magnification of the optical system is at least fivefold and the optical system has a zoom function and/or a magnification of the surroundings camera is at most fivefold.

26. The surgical assistance system according to claim 15, wherein the microscope head comprises the camera system and is adapted in such a way via an orientation of its optical camera axis and via its camera optics that a field of view of the surrounding camera includes the field of view of the optical system.

27. The surgical assistance system according to claim 15, wherein the surgical assistance system is adapted to spatially detect a face of the patient as the 3D structure.

28. The surgical assistance system according to claim 15, wherein only the surrounding camera is used to correlate the 3D structure with the preoperative 3D image data to register the patient.

29. The surgical assistance system according to claim 15, wherein the surrounding camera is arranged rigidly on the housing.

30. An image display method for correlated display of a microscope image and a surroundings image for the surgical assistance system according to claim 15, comprising the steps of:
A. targeting an area via the optical system arranged on the microscope head;
B. creating the microscope image by the downstream microscope image unit of a magnification provided by the optical system;
C. creating the surroundings image by the at least one surroundings camera;
D. creating the combined view with the microscope image and the surroundings image;
E. outputting the combined view by the display device;
F. spatially detecting the 3D structure via the camera system; and
G. correlating the 3D structure with the 3D image data to register the patient.

31. The image display method according to claim 30 further comprising the step of arranging the at least one surroundings camera on the microscope head.

32. A computer-readable storage medium comprising instructions which, when executed by a computer, cause the computer to perform the steps of claim 30.

33. A medical sterile space comprising the surgical assistance system according to claim 15.

34. A surgical assistance system for use in a surgical intervention on a patient, the surgical assistance system comprising:
A. a surgical microscope comprising:
i. a microscope head that is movable, the microscope head comprising a housing and an optical system adapted to provide optical magnification of a targeted area in a direction of an optical microscope axis and to create a microscope image via a downstream microscope image unit;
ii. a microscope arm that is movable and connected to a base, the microscope head being attached on the base, the base being adapted to adjust a position and/or orientation of the microscope head; and
iii. a camera system with at least one surrounding camera arranged on the microscope head, the at least one surrounding camera being adapted in such a way that a field of view of the at least one surrounding camera includes a field of view of the optical system to detect an area targeted by the optical system and surroundings around the targeted area and to provide a surroundings image;
B. at least one display device for displaying a visual content;
C. a control unit adapted to:
i. process the microscope image and control the at least one display device for a view accordingly, and
ii. process the surroundings image and generate a combined view with the microscope image and the surroundings image and output the combined view visually by the display device; and
D. at least one actuator to actively move the microscope arm and the microscope head,
the control device being adapted to control the actuator to actively move the microscope head to a predetermined position and/or orientation.
